(19) United States
(12) Patent Application Publication
(10) Pub. No.: US 2026/0130664 A1
(43) Pub. Date: May 14, 2026

(54) SLIP FREE ENGAGEMENT BETWEEN NEEDLE AND NEEDLE DRIVER JAWS

(71) Applicant: CILAG GMBH INTERNATIONAL, Zug (CH)

(72) Inventors: Konstantin G. ZABOTKIN, Mason, OH (US); Alexander Michael CANNARA, Roseland, NJ (US)

(73) Assignee: CILAG GMBH INTERNATIONAL, Zug (CH)

(21) Appl. No.: 19/379,391

(22) Filed: Nov. 4, 2025

Related U.S. Application Data
(60) Provisional application No. 63/718,016, filed on Nov. 8, 2024.

(51) Int. Cl. A61B 17/06; A61B 17/04; A61B 17/29

(52) U.S. Cl. CPC A61B 17/06066; A61B 17/0469; A61B 17/29; A61B 2017/2926

(57) ABSTRACT

A surgical tool is disclosed that includes an end effector providing opposing first and second jaws, opposing first and second grips provided on the first and second jaws, respectively, and a suturing needle defining grooves sized to receive the first and second grips of the end effector.

### CROSS-REFERENCE TO RELATED APPLICATIONS

The present application claims priority under 35 U.S.C. §119(e) to U.S. Provisional Application No. 63/718,016, titled “SLIP FREE ENGAGEMENT BETWEEN NEEDLE AND NEEDLE DRIVER JAWS”, filed November 8, 2024, the disclosure of which is hereby incorporated by reference in its entirety.

### BACKGROUND

The present disclosure is related to surgical tools and, more specifically, to surgical tool needle drivers for gripping a needle to place sutures during a surgical procedure.

During a surgical procedure, a needle driver of a robotic surgical tool is commonly used to grip a suturing needle to apply sutures to a patient. While placing sutures, the grip of the jaws of the needle driver on the suturing needle must remain secure and consistent. However, the amount of grip force that the needle driver jaws of the robotic surgical tool can apply to a needle is significantly less compared to the amount of grip force that needle driver jaws of a handheld surgical tool can apply to a needle.

Accordingly, an improved needle and/or needle driver jaws is desired to improve engagement between the needle and the needle driver without increasing grip strength.

### BRIEF DESCRIPTION OF THE DRAWINGS

The following figures are included to illustrate certain aspects of the present disclosure, and should not be viewed as exclusive embodiments. The subject matter disclosed is capable of considerable modifications, alterations, combinations, and equivalents in form and function, without departing from the scope of this disclosure.

FIG. 1 shows a diagram of an example surgical tool that may incorporate certain principles of the present disclosure.

FIG. 2 shows a diagram illustrating the degrees of freedom through which a wrist of a surgical tool may articulate.

FIGS. 3-5 show various views of an end effector of the example surgical tool of FIG. 1.

FIG. 6 shows a diagram illustrating coupling between a surgical tool and a robotic manipulator.

FIG. 7 is an enlarged isometric view of a traditional arrangement of the end effector of the surgical tool of FIG. 1 gripping a suturing needle.

FIG. 8 is an enlarged side view of the end effector of FIG. 7 gripping the suturing needle.

FIG. 9 is an enlarged isometric view of an improved end effector for use with the surgical tool of FIG. 1 gripping an improved suturing needle, in accordance with at least one aspect of the present disclosure.

FIG. 10 is an enlarged side view of the improved end effector of FIG. 9 gripping the improved suturing needle, in accordance with at least one aspect of the present disclosure.

FIGS. 11A-11C are various loading modes on the improved suturing needle of FIG. 9 while being gripped by the improved end effector of FIG. 9, in accordance with at least one aspect of the present disclosure.

### DETAILED DESCRIPTION

The present disclosure is related to surgical tools and, more specifically, to surgical tool needle drivers for gripping a needle to place sutures during a surgical procedure.

The terms “proximal” and “distal” are defined herein relative to the location of engagement by a surgeon or a robotic manipulator. The term “proximal” refers to a position closer to the location of engagement (i.e., further away from a patient), and the term “distal” refers to a position more removed from the location of engagement (i.e., nearer to a patient). Moreover, directional terms such as above, below, upper, lower, upward, downward, left, right, and the like are used to describe relative position in the figures and thus should not be considered limiting.

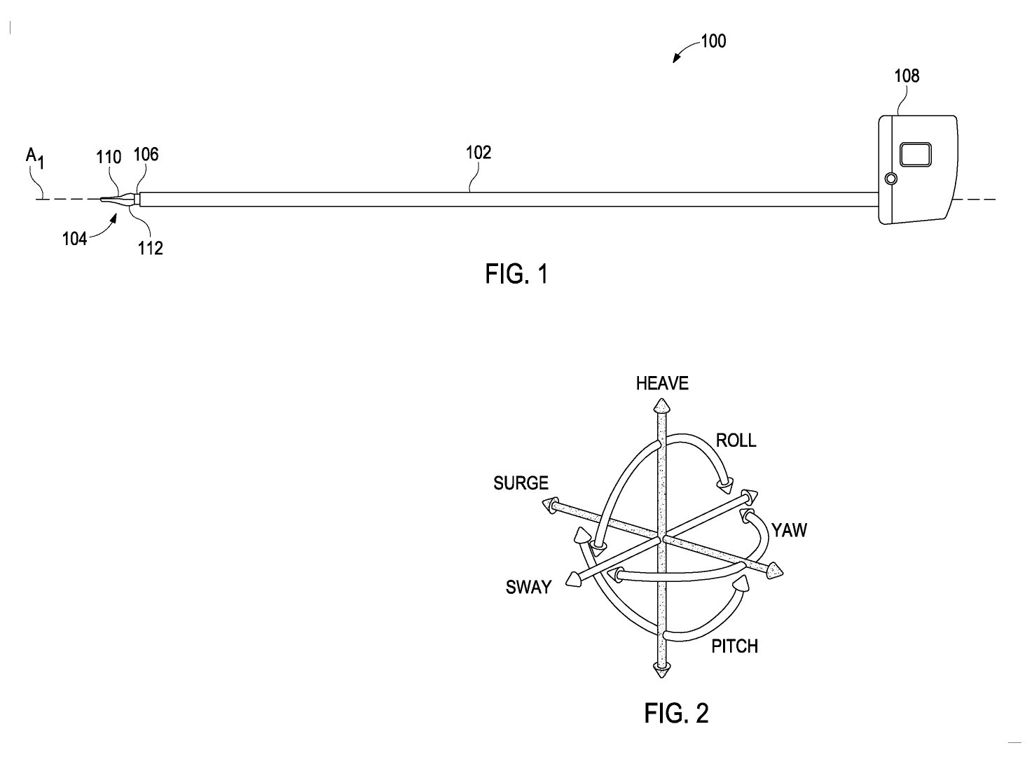

FIG. 1 shows a diagram of an example robotic surgical tool 100 that may incorporate certain principles of the present disclosure. The robotic surgical tool 100 includes an elongate shaft 102, an end effector 104 located at a distal end of the elongate shaft 102, and a housing 108 located at a proximal end of the elongate shaft 102. The wrist 106 is also located at a distal end of the elongate shaft 102 and couples the end effector 104 thereto. The housing 108 may be configured for releasable coupling with a mounting fixture of a robotic manipulator, alternately referred to as a “robot” or “surgical robot.” The housing 108 contains various mechanisms (obscured in FIG. 1) which may be actuated to produce one or more resultant motions in the end effector 104. More particularly, actuation within the housing 108 controls the operation of the end effector 104 via retraction and extension of cables or similar elongate members (obscured in FIG. 1) that are operably engaged with the end effector 104.

The housing 108 may be releasably coupled with the mounting fixture of a robotic manipulator in a variety of ways, such as by clamping or clipping thereto, or slidably mating therewith. Illustrative mechanisms for releasably coupling the housing 108 to a mounting fixture are described in more detail in U.S. Patent Nos. 8,831,782, 9,884,427 and 10,149,726, as well as U.S. Patent Publication No. 2015/0025549, all of which are incorporated by reference in their entirety herein.

Continuing with FIG. 1, the end effector 104 is configured to move relative to the elongate shaft 102 at the wrist 106, such as by pivoting at the wrist 106, to position the end effector 104 at a desired orientation and location relative to a surgical site during a surgical procedure. The housing 108 includes various components designed to position and operate various features of the end effector 104 (e.g., one or more of clamping, firing, rotation, articulation, energy delivery, and the like). In illustrative embodiments, one or more elongate members extend from the housing 108 through the wrist 106 to facilitate articulation of the end effector 104, as discussed in more detail herein. In at least some embodiments, the elongate shaft 102 and the end effector 104 are coupled distally thereto are configured to rotate about a longitudinal axis A₁. In some embodiments, various components of the housing 108 can be configured to facilitate rotational motion of the elongate shaft 102 and the end effector 104 about the longitudinal axis A₁. In other embodiments, the elongate shaft 102 may be fixed to the housing 108, in which case the robotic surgical tool 100 may be rotated by the robotic manipulator to reposition the elongate shaft 102 and the end effector 104.

The robotic surgical tool 100, particularly at the end effector 104, can be configured to perform at least one surgical function. The choice of the end effector 104 can determine which surgical function the robotic surgical tool 100 is able to perform. Illustrative configurations of the end effector 104 that may be present in the robotic surgical tool 100 include, for example, forceps, graspers, needle drivers, scissors, electrocauterization tools that apply energy to tissue, staplers, clip appliers, suctioning tools, irrigation tools, imaging devices (e.g., endoscopes or ultrasonic probes), and any combination thereof. In at least one embodiment, the robotic surgical tool 100 may be configured to apply mechanical force to a tissue. The mechanical force can be conveyed to the end effector 104 via the cables or similar elongate members extending through the elongate shaft 102.

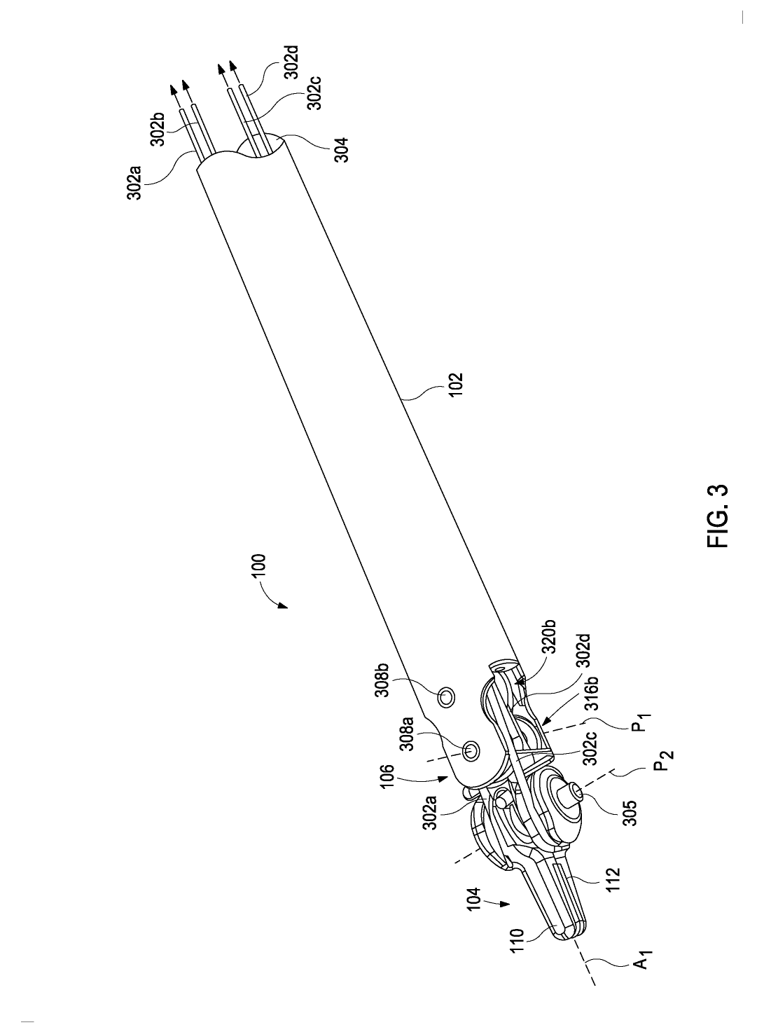

The elongate shaft 102 extends distally from the housing 108 and has at least one lumen (see FIG. 3) extending internally therethrough. The elongate shaft 102 may be affixed to the housing 108, but alternately may be releasably coupled so as to be interchangeable with other types of elongate shafts, such as elongate shafts having a differing diameter. In at least some embodiments, the elongate shaft 102 may be rotatably coupled to the housing 108.

The end effector 104 can have a variety of sizes, shapes and configurations. In the illustrative configuration of FIG. 1, the end effector 104 comprises a tissue grasper or “needle driver” having opposing jaws 110 and 112 that are configured to move (pivot) relative to one another between open and closed positions. In addition, the entirety of the end effector 104 may pivot relative to the elongate shaft 102 at the wrist 106. Pivoting may place the end effector 104 in a desired position to engage tissue or another surface during a surgical procedure.

The wrist 106 can likewise have a variety of configurations. In the illustrative configuration of FIG. 1, the wrist 106 includes a joint configured to allow movement of the end effector 104 relative to the elongate shaft 102, such as a pivot joint at which the jaws 110 and 112 are pivotally attached via a corresponding body. Illustrative configurations that may be similar to the wrist 106 and are suitable for use in the embodiments of the present disclosure include those described in U.S. Patent Nos. 8,831,782, 9,884,427 and 10,149,726, as well as U.S. Patent Publication No. 2015/0025549, all of which were previously incorporated by reference in their entirety above.

FIG. 2 shows a diagram illustrating the degrees of freedom through which wrist 106 may articulate. More specifically, the degrees of freedom available to the wrist 106 are represented by three translational or position variables (e.g., surge, heave and sway) and three rotational or orientation variables (e.g., Euler angles or roll, pitch and yaw). The translational and rotational variables collectively describe the position and orientation of one or more components of a surgical system (e.g., the wrist 106 and the associated end effector 104) with respect to a given frame of reference, such as a Cartesian coordinate system or spherical coordinate system. As illustrated in FIG. 2, the term “surge” refers to forward and backward movement, the term “heave” refers to up and down movement, and the term “sway” refers to left and right movement. With regard to the rotational terms in FIG. 2, “roll” refers to side-to-side tilting, “pitch” refers to forward and backward tilting, and “yaw” refers to left and right turning.

In some embodiments, a pivoting motion can include pitch movement about a first axis of the wrist 106 (e.g., X-axis), yaw movement about a second axis of the wrist 106 (e.g., Y-axis), and combinations thereof to allow for 360° rotational movement of the end effector 104 about the wrist 106. In other embodiments, a pivoting motion can be limited to movement in a single plane such that the end effector 104 rotates only in a single plane (e.g., only pitch movement about a first axis of the wrist 106 or only yaw movement about a second axis of the wrist 106).

The robotic surgical tool 100 includes a plurality of cables or similar elongate members (obscured in FIG. 1), which are configured to impart movement to the end effector 104 relative to the elongate shaft 102. Illustrative forms of the elongate members include, for example, cables, bands, lines, cords, wires, ropes, strings, twisted strings and the like. Elongate members can be formed from any of a variety of high-durability materials, such as a metal (e.g., tungsten, stainless steel, and like materials) or a polymer. In at least one embodiment, one or more of the elongate members may be made of a flexible material. Illustrative cables and similar elongate members are described in U.S. Patent Nos. 8,831,782, 9,884,427 and 10,149,726, as well as U.S. Patent Publication No. 2015/0025549, all of which were previously incorporated by reference in their entirety above.

The disposition of the elongate members within the robotic surgical tool 100 is illustrated more fully in FIGS. 3-5, which show various enlarged views of the elongate shaft 102, the end effector 104, and the wrist 106. Although the robotic surgical tool 100 is depicted as including four elongate members 302a-d, one pair being operatively coupled to each of the jaws 110 and 112, alternative configurations can have differing numbers of elongate members. For example, a surgical tool having an end effector that does not require internal motion can include two elongate members configured to provide articulation upon longitudinal tensioning and de-tensioning.

Referring first to FIG. 3, elongate members 302a-d extend longitudinally within a lumen 304 of the elongate shaft 102 through the wrist 106 and operably engage the end effector 104, as described hereinafter. The proximal ends of elongate members 302a-d are similarly operably engaged with components in the housing 108 (FIG. 1). One or more of the elongate members 302a-d may be selectively translated longitudinally to cause the end effector 104 to move (e.g., pivot in one or more locations) relative to elongate shaft 102. Depending on the required motion, one or more of the elongate members 302a-d may translate longitudinally to articulate the end effector 104 (e.g., to move the jaws 110, 112 at an angle in a same direction), or open and close the jaws 110, 112, or any combination thereof.

Although a single lumen 304 is depicted in FIG. 3, multiple lumens can be present in alternative embodiments, such that one or more of the elongate members 302a-d is housed within each of the multiple lumens. In further alternative embodiments, one or more of the elongate members 302a-d can extend along the exterior of the elongate shaft 102, such as in longitudinal channels formed in an exterior surface of the elongate shaft 102.

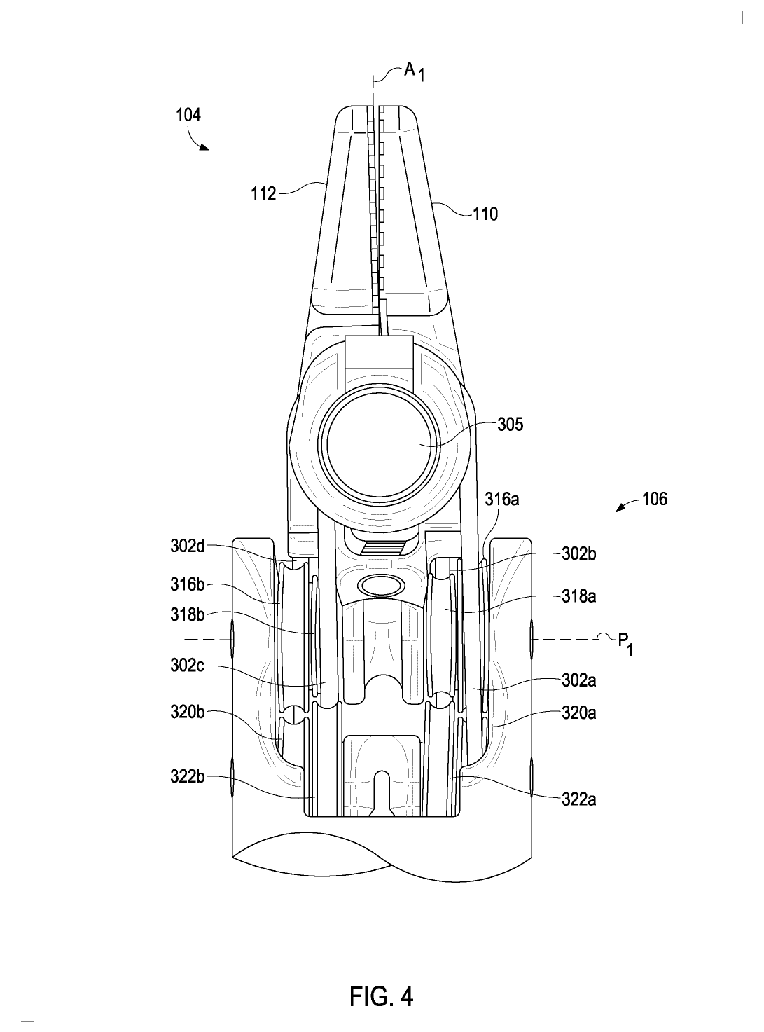

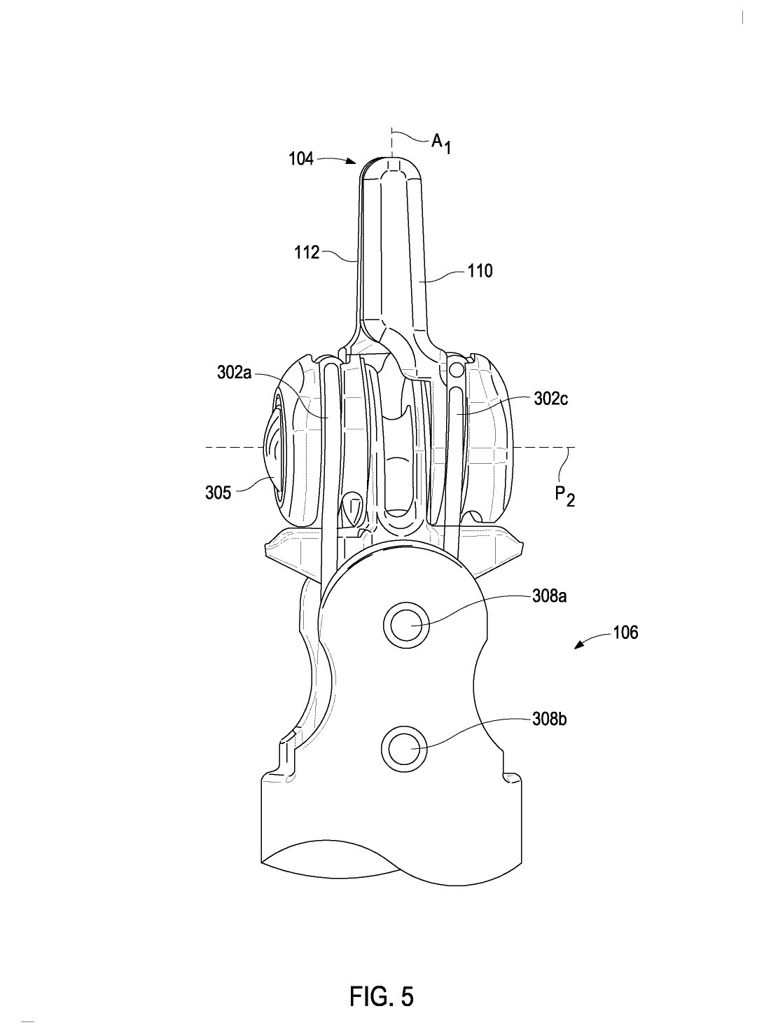

Referring still to FIG. 3, and with further reference to FIGS. 4 and 5, the wrist 106 includes multiple pulleys for engaging and redirecting elongate members the 302a-d during their longitudinal translation. Specifically, the wrist 106 includes a distal plurality of pulleys 316a, 316b, 318a and 318b, and a proximal plurality of pulleys 320a, 320b, 322a and 322b. As best seen in FIG. 4, clearance is provided between corresponding pulleys in the distal and proximal pluralities of pulleys, which is sized for passage of the elongate members 302a-d therethrough. The pulleys 316a,b and 318a,b are mounted to a distal wrist axle 308a, and the pulleys 320a,b, and 322a,b are mounted to a proximal wrist axle 308b. The end effector 104 is operably coupled to the wrist 106 such that the distal wrist axle 308a defines first pivot axis P₁ during operation thereof.

The robotic surgical tool 100 further includes a second pivot axis P₂ along an end effector axle 305, about which the jaws 110 and 112 are configured to pivot relative to each other from a closed position through a range of open positions, and/or about which the jaws 110 and 112 are configured to move together during articulation of end effector 104. As illustrated, the second pivot axis P₂ is substantially perpendicular to the longitudinal axis A₁. A person having ordinary skill in the art will appreciate that axes the A₁ and P₂ may not be precisely perpendicular to one another but nevertheless be considered to be substantially perpendicular due to any number of factors, such as manufacturing tolerance and precision of measurement devices.

The robotic surgical tool 100 has two joints at the second pivot axis P₂, one joint for each of the jaws 110 and 112. Actuation of at least one of the elongate members 302a-d causes movement of the jaw 110 and/or jaw 112 at the associated joint(s) along the second pivot axis P₂. In an example embodiment, the jaws 110 and 112 are configured to pivot in tandem at their associated joints. That is, during opening of the jaws 110 and 112, each of the jaws 110 and 112 rotates at its associated joint, and during closing of the jaws 110 and 112, each of the jaws 110 and 112 rotates in the opposite direction at its associated joint.

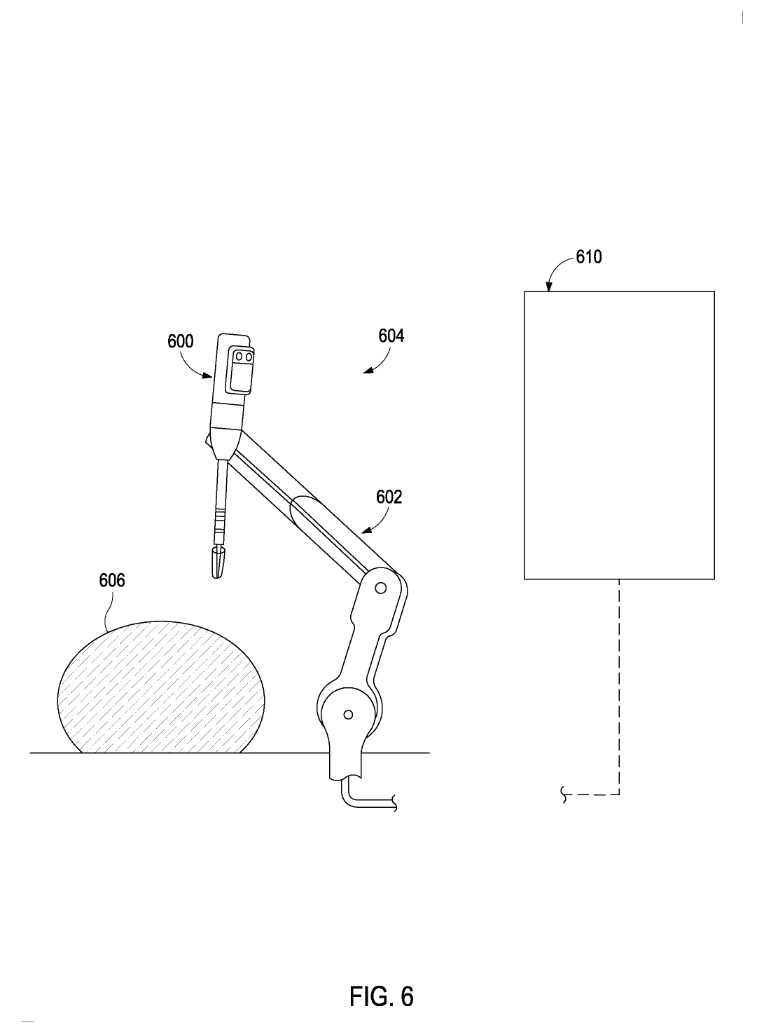

In at least some embodiments, the robotic surgical tool 100 is configured for releasable coupling to a robotic manipulator. FIG. 6 shows a diagram illustrating coupling between a surgical tool and a robotic manipulator. It is to be understood that the manner of coupling depicted in FIG. 6 is illustrative in nature so that certain embodiments of the present disclosure can be better understood. In non-limiting variations, the type of surgical tool and/or robotic manipulator, and/or the manner of coupling, for example, may differ based upon considerations that will be familiar to one having ordinary skill in the art.

As depicted in FIG. 6, a robotic surgical tool 600 is coupled to an arm 602 of a robotic manipulator 604. The robotic surgical tool 600 may be similar to the robotic surgical tool 100 (FIG. 1). The robotic manipulator 604 and the robotic surgical tool 600 are positioned adjacent to a patient 606 in order to conduct a surgical procedure thereon. The robotic manipulator 604 is in electronic communication with a control system 610, examples of which are described in U.S. Patent No. 11,424,027, which is hereby incorporated by reference in its entirety herein, through which a surgeon may move the arm 602 and/or actuate the robotic surgical tool 600 according to one or more embodiments. Although FIG. 6 has depicted a wired connection between the robotic surgical tool 600 and the control system 610, wireless configurations also reside within the scope of the present disclosure. In one or more embodiments, the control system 610 may include vision control, processing control, or any combination thereof, using any combination of software and hardware implementation.

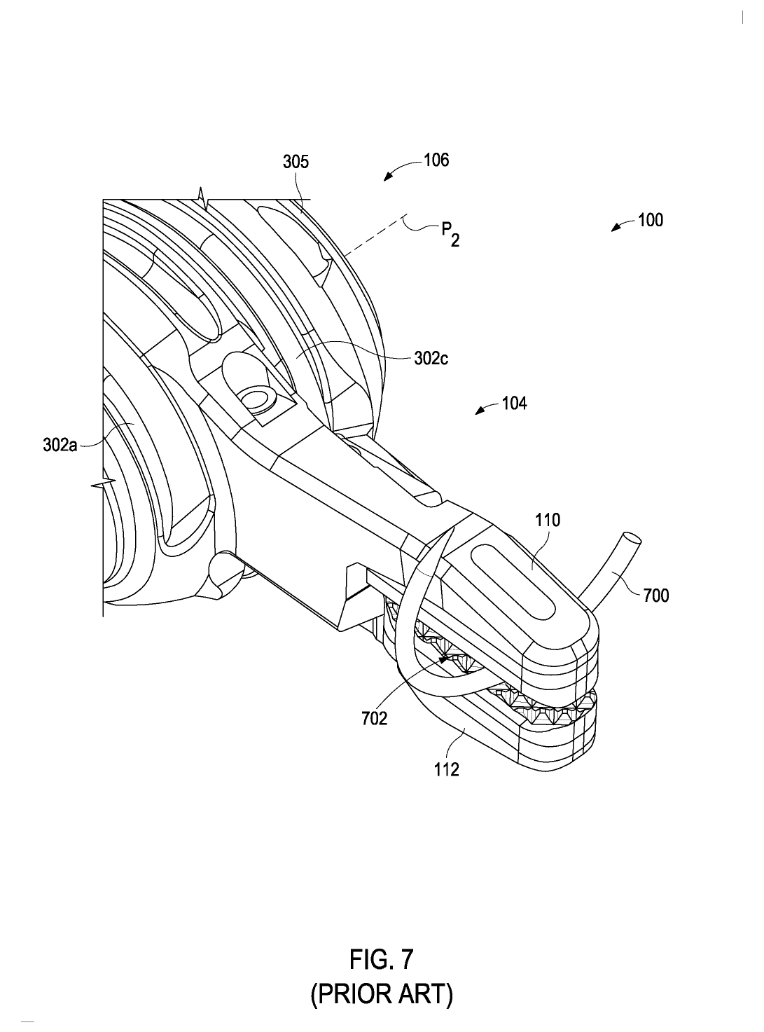

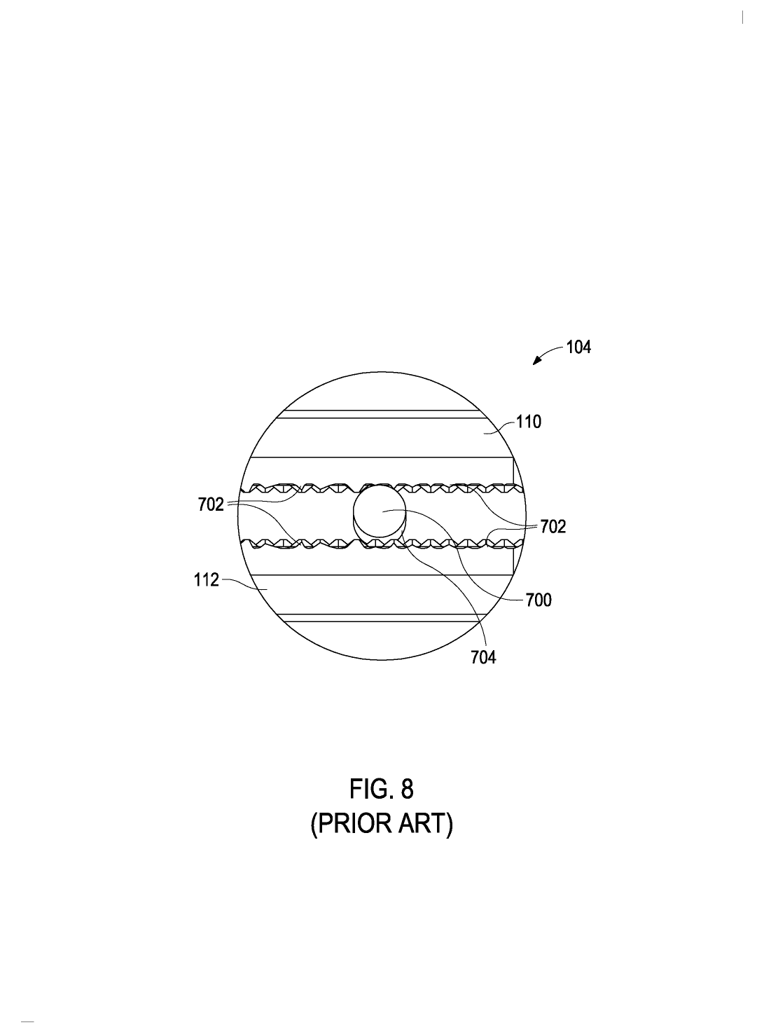

FIGS. 7 and 8 illustrate a traditional arrangement of the end effector 104 gripping a suturing needle 700. During a surgical procedure, the end effector 104 of the robotic surgical tool 100 may grip (grasp) a suturing needle 700 such that the robotic surgical tool 100 can apply sutures to the patient 606 (FIG. 6). More specifically, the jaws 110, 112 of the end effector 104 may each provide (define) opposing grips 702 while the suturing needle 700 may provide a smooth outer surface profile 704 that is graspable by the grips 702 of the jaws 110, 112.

While placing sutures, the grip of the end effector 104 onto the suturing needle 700 (e.g. the engagement between the grips 702 and the outer surface profile 704 of the suturing needle 700) must remain secure and consistent. However, the amount of grip force that the end effector 104 of the robotic surgical tool 100 can apply to the suturing needle 700 is significantly less compared to the amount of grip force that needle driver jaws of a handheld surgical tool can apply to a needle. Due to the reduced grip strength, the grips 702 may not be able to provide the necessary grip strength to the smooth outer surface profile 704 of the suturing needle 700 to maintain the lateral position, the longitudinal position, and/or the rotational orientation of the suturing needle 700, thereby keeping the suturing needle 700 secure and consistent during the surgical procedure. Accordingly, an improved needle and/or end effector is desired that improves engagement between the suturing needle and the end effector without increasing grip strength.

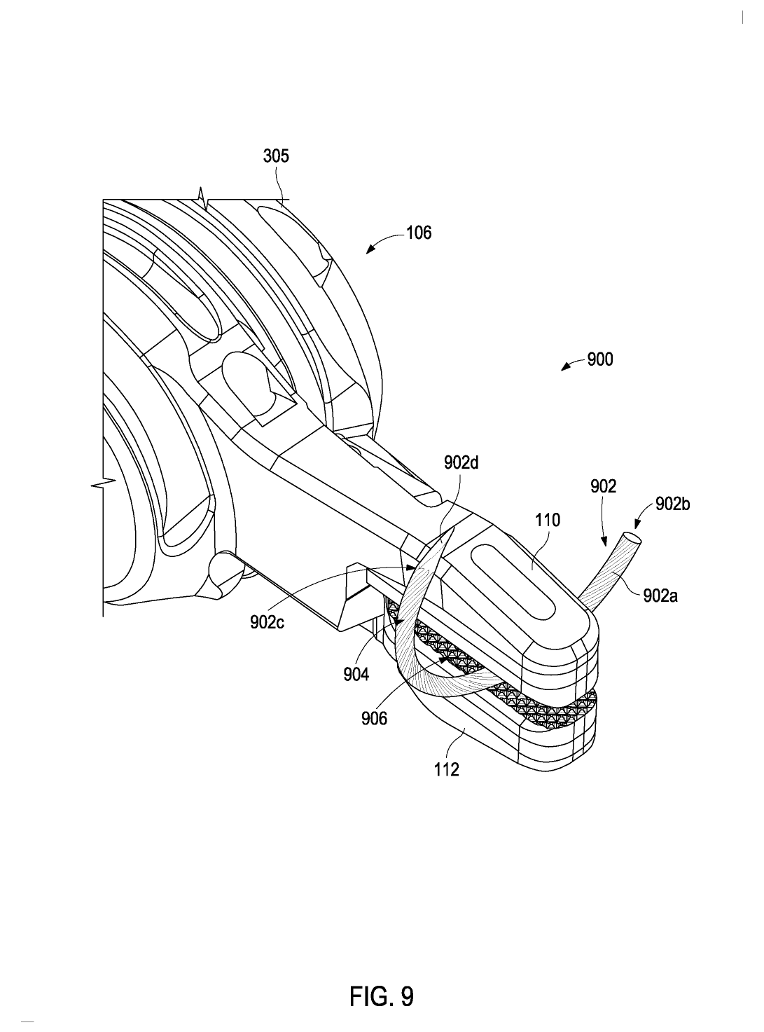

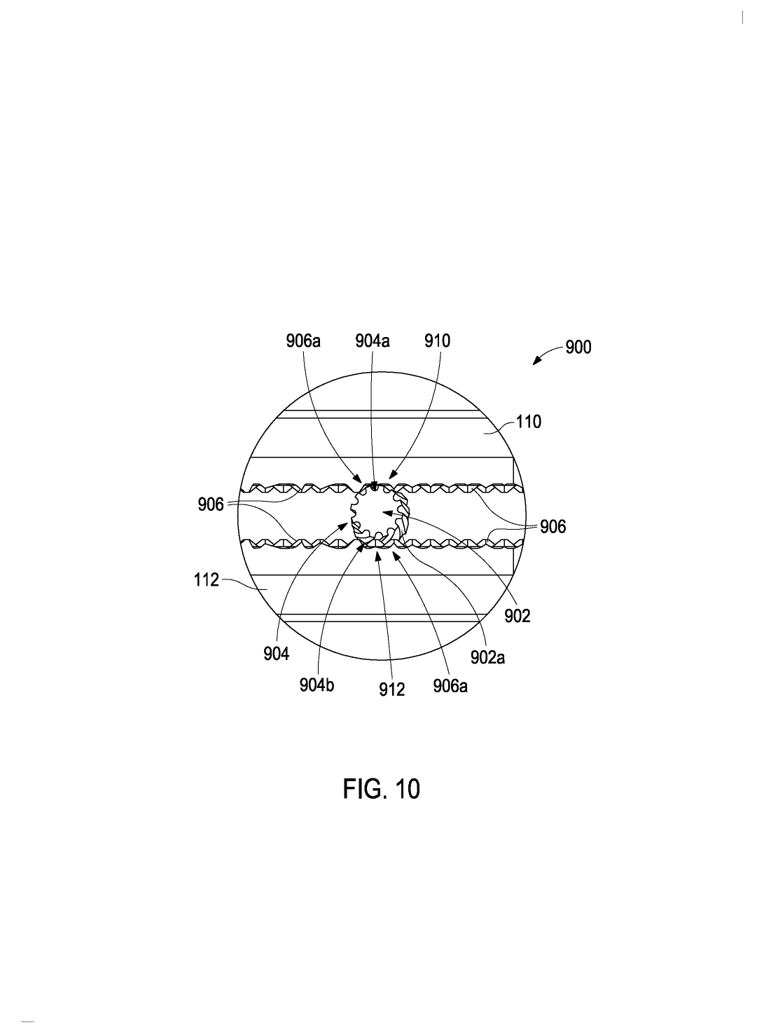

FIGS. 9 and 10 provide an improved end effector 900 and an improved suturing needle 902, in accordance with at least one aspect of the present disclosure. The end effector 900 may be similar in some respects to the end effector 104 of FIG. 1 and therefore may be best understood with reference thereto, where like numerals will correspond to like components not described again in detail. The end effector 900 and the suturing needle 902 may be used in lieu of the end effector 104 (FIG. 1) and the suturing needle 700 (FIG. 7) with either of the robotic surgical tools 100, 600 (FIGS. 1 and 6, respectively).

The suturing needle 902 may include an outer surface 902a that provides (defines) a plurality of grooves 904. The grooves 904 may be formed in the outer surface 902a via a knurling process or any other suitable process, such as with a laser, a casting process, machining, die forming, die drawing, stamping, milling, or grinding, or combinations thereof. The grooves 904 serve to increase the surface area of the suturing needle 902 compared to traditional suturing needles that include smooth outer surface profiles, like suturing needle 700 (FIG. 7) with smooth outer surface profile 704 (FIG. 7).

In some embodiments, as illustrated in FIGS. 9 and 10, the grooves 904 may extend along the length of the suturing needle 902 from a first or “proximal” end 902b of the suturing needle to a second or “distal” end 902c of the suturing needle 902 that is adjacent to a tip 902d of the suturing needle 902. Having grooves 904 that span the length of the suturing needle 902 enables a user to grasp the suturing needle 902 with the jaws 110, 112 at multiple locations along the length thereof. In other embodiments, the grooves 904 extend along only a portion of the suturing needle 902, less than the length of the suturing needle 902. The length of the portion may be the same, or similar, to the width of the first and second jaws 110, 112. Having grooves 904 that span only a portion of the suturing needle 902 may provide a predefined location for a user to grasp the suturing needle 902 with the jaws 110, 112.

In some embodiments, as illustrated in FIGS. 9 and 10, the grooves 904 may be helical grooves that extend radially and longitudinally along the length of the suturing needle 902. The helical grooves may be formed (defined) in the outer surface 902a in a first radial direction along the length of the suturing needle 902 (e.g. clockwise direction) or a second radial direction along the length of the suturing need 902 opposite the first radial direction (e.g. counterclockwise direction), or combinations thereof. In other embodiments, the grooves 904 are linear grooves that extend linearly along the length of the suturing needle 902. The quantity and orientation of the grooves 904 may vary to increase the surface area of the suturing needle 902 depending on the intended surgical purpose of the suturing needle 902. The suturing needle 902 may further define one or more cavities (not shown) along the length thereof. The cavities may be defined within the grooves 904 or in the outer surface 902a at locations that lack grooves 904.

Each of the grooves 904 may exhibit a first shape. In some embodiments, as best seen in FIG. 10, the first shape may be a semi-circular shape. In other embodiments, the first shape is a square shape, a rectangular shape, a triangular shape, or a trapezoidal shape, or combinations thereof.

One, or both, of the jaws 110, 112 of the end effector 900 may include grips 906 that are sized to be at least partially, or completely, received in corresponding grooves 904 of the suturing needle 902. When the grips 906 are received in the grooves 904, a mechanical locking interface may be established (defined) between the jaws 110, 112 and the suturing needle 902 that prevents, counteracts, or at least substantially inhibits, the suturing needle 902 from moving relative to the end effector 900, as will be explained in more detail below. The locking interface may include a first locking interface 910 that is defined between a first portion 906a of grips 906 of the first jaw 110 being received in a first portion 904a of grooves 904 of the suturing needle 902 and a second locking interface 912 that is defined between a second portion 906b of grips 906 of the second jaw 112 being received in a second portion 906b of the grooves 904 of the suturing needle 902.

The first locking interface 910 may provide multiple (i.e., two or more) lock points between the first jaw 110 and the suturing needle 902 and the second locking interface 912 may similarly provide multiple (i.e., two or more) lock points. A ”lock point” may be defined as interface between a grip 906 and a corresponding groove 904 once the grip 906 has been received in the groove 904. This contrasts with traditional end effector and suturing needle arrangements (e.g., the end effector 104 (FIG. 1) and the suturing needle 700 (FIG. 7)), which lack any locking interfaces or lock points due to the lack of grooves defined in the suturing needle 700.

The grips 906 may exhibit a second shape. In some embodiments, the second shape may be a pyramidal shape, as illustrated in FIGS. 9 and 10. In other embodiments, the second shape may be a conical shape, a rectangular shape, a cubical shape, or a cylindrical shape, or combinations thereof. The second shape may be the same or different than the first shape. The grips 906 may be formed on the first and second jaws 110, 112 via a knurling process or any other suitable process, such as with an injection molding process, a laser, a casting process, machining, die forming, die drawing, stamping, milling, or grinding. The first and second jaws 110, 112 may be a first material (e.g., metal) and the grips 906 may be a second material different than the first material (e.g., a polymer or a hard material with an HRC of 60 or higher, like tungsten carbide, to assure a reliable engagement to the suturing needle 902). Alternatively, the first and second jaws 110, 112 and the grips 906 may be the same material.

The number of grooves 904 and grips 906 may be selected based on a desired number of points of contact between the end effector 900 and the suturing needle 902. For example, increasing the number of grooves 904 and grips 906 may increase the mechanical interlocking interface between the end effector 900 and the suturing needle 902, while decreasing the number of grooves 904 and grips 906 may decrease the mechanical interlocking interface between the end effector 900 and the suturing needle 902. Accordingly, the amount of the locking forces between the end effector 900 and the suturing needle 902 can be adjusted.

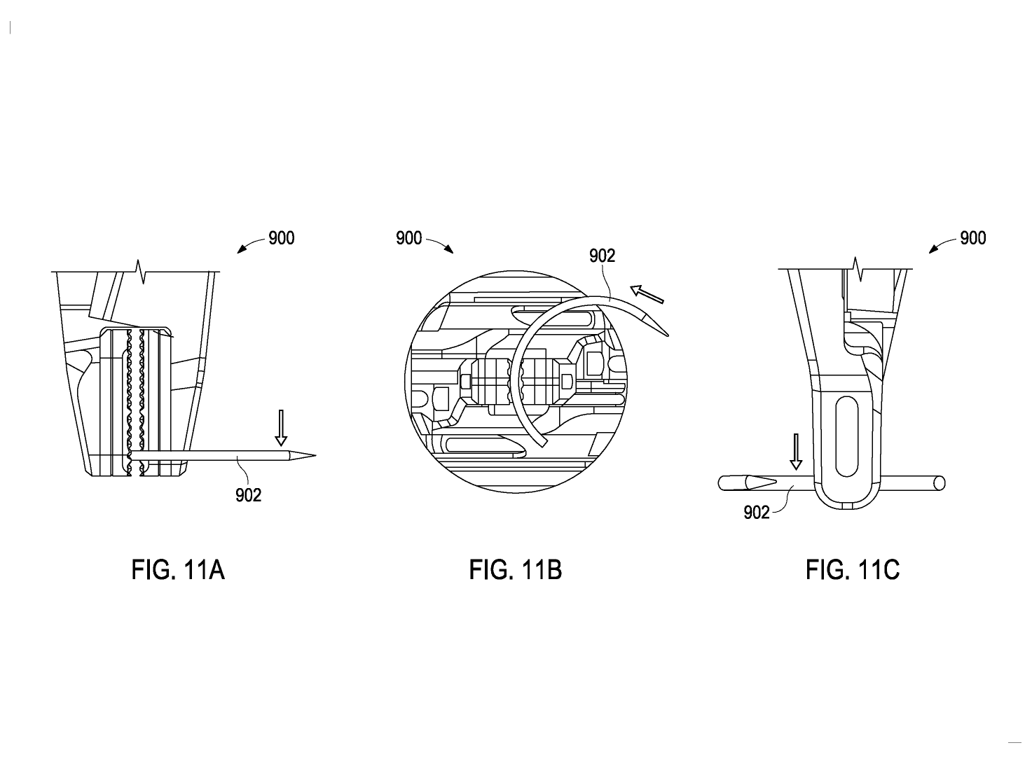

The mechanical locking interface provides a secure and consistent grip between the end effector 900 and the suturing needle 902, regardless of the grip force provided by the jaws 110, 112. For instance, referring to FIG. 11A, the mechanical locking interface defined by the grips 906 being received in the grooves 904 may prevent, or at least substantially inhibit or counteract, a first degree of motion, which may be rotation of the tip 902d of the suturing needle 902 relative to (toward or away from) the jaws 110, 112, thereby “locking” a radial (rotational) position of the suturing needle 902 relative to the end effector 900. In addition, referring to FIG. 11B, the mechanical locking interface defined by the grips 906 being received in the grooves 904 may prevent, or at least substantially inhibit or counteract, a second degree of motion, which may be lateral movement of the suturing needle 902 relative to the jaws 110, 112, thereby “locking” a lateral position of the suturing needle 902 relative to the end effector 900. In addition, referring to FIG. 11C, the mechanical locking interface defined by the grips 906 being received in the grooves 904 may prevent, or at least substantially inhibit or counteract, a third degree of motion, which may be longitudinal movement of the suturing needle 902 relative to the jaws 110, 112, thereby “locking” a longitudinal position of the suturing needle 902 relative to the end effector 900. Accordingly, the mechanical locking interface between the end effector 900 and the suturing needle 902 may lock multiple degrees of motion, thereby creating a more reliable and consistent position of the suturing needle 902 during a surgical procedure.

The improved end effector 900 and suturing needle 902 may be used independently to improve the grip strength between end effectors and suturing needles. For instance, end effector 900 may be used with suturing need 700 (FIG. 7) and end effector 104 (FIG. 1) may be used with suturing needle 902. However, using the end effector 900 and suturing needle 902 together creates a more reliable and consistent “locking” effect compared to alternative end effectors and needles.

Embodiments disclosed herein include:

A. A surgical tool comprising an end effector providing opposing first and second jaws, opposing first and second grips provided on the first and second jaws, respectively, and a suturing needle defining grooves sized to receive the first and second grips of the end effector.

B. A surgical tool comprising an end effector providing opposing first and second jaws, grips provided on the first jaw, and a suturing needle defining grooves sized to receive the grips of the first jaw.

C. A surgical tool comprising an end effector providing opposing first and second jaws and a suturing needle graspable by the end effector, wherein a first locking interface is defined when the end effector grasps the suturing needle and a second locking interface is defined when the end effector grasps the suturing needle, wherein the first and second locking interfaces co-operatively oppose multiple degrees of motion of the suturing needle relative to the end effector.

Each of embodiments A, B, and C may have one or more of the following additional elements in any combination: Element 1: wherein a longitudinal position of the suturing needle is locked relative to the end effector when the first and second grips are received in the grooves. Element 2: wherein a lateral position of the suturing needle is locked relative to the end effector when the first and second grips are received in the grooves. Element 3: wherein a radial position of the suturing needle is locked relative to the end effector when the first and second grips are received in the grooves. Element 4: wherein the grooves extend longitudinally and radially along a length of the suturing needle. Element 5: wherein the first and second grips comprise pyramidal grips. Element 6: wherein a locking interface is defined between the first jaw and the suturing needle when the grips are received in the grooves. Element 7: wherein the grips are first grips and the surgical tool further comprises second grips provided on the second jaw. Element 8: wherein a first locking interface is defined between the first jaw and the suturing needle when the first grips are received in a first portion of the grooves and a second locking interface is defined between the second jaw and the suturing needle when the second grips are received in a second portion of the grooves. Element 9: wherein the first and second locking interfaces co-operatively lock a longitudinal position of the suturing needle relative to the end effector. Element 10: wherein the first and second locking interfaces co-operatively lock a lateral position of the suturing needle relative to the end effector. Element 11: wherein the first and second locking interfaces co-operatively lock a rotational position of the suturing needle relative to the end effector. Element 12: wherein the grooves extend radially and longitudinally along a length of the suturing needle. Element 13: wherein the first and second locking interfaces co-operatively lock a longitudinal position of the suturing needle relative to the end effector. Element 14: wherein the first and second locking interfaces co-operatively lock a lateral position of the suturing needle relative to the end effector. Element 15: wherein the first and second locking interfaces co-operatively lock a rotational position of the suturing needle relative to the end effector. Element 16: wherein the first jaw provides first grips and the suturing needle provides first grooves to receive the first grips to define the first locking interface and the second jaw provides second grips and the suturing needle provides second grooves to receive the second grips to define the second locking interface. Element 17: wherein the first and second grooves extend radially and longitudinally along a length of the suturing needle.

By way of non-limiting example, exemplary combinations applicable to A, B, and C include: Element 7 with Element 8; Element 7 with Elements 8 and 9; Element 7 with Elements 8 and 10; Element 7 with Elements 8 and 11; Element 16 with Element 17.

Therefore, the disclosed systems and methods are well adapted to attain the ends and advantages mentioned as well as those that are inherent therein. The particular embodiments disclosed above are illustrative only, as the teachings of the present disclosure may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope of the present disclosure. The systems and methods illustratively disclosed herein may suitably be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the elements that it introduces. If there is any conflict in the usages of a word or term in this specification and one or more patent or other documents that may be incorporated herein by reference, the definitions that are consistent with this specification should be adopted.

As used herein, the phrase “at least one of” preceding a series of items, with the terms “and” or “or” to separate any of the items, modifies the list as a whole, rather than each member of the list (i.e., each item). The phrase “at least one of” allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

The use of directional terms such as above, below, upper, lower, upward, downward, left, right, and the like are used in relation to the illustrative embodiments as they are depicted in the figures, the upward direction being toward the top of the corresponding figure and the downward direction being toward the bottom of the corresponding figure.

What is claimed is:

1. A surgical tool, comprising:
an end effector providing opposing first and second jaws;
opposing first and second grips provided on the first and second jaws, respectively; and
a suturing needle defining grooves sized to receive the first and second grips of the end effector.

2. The surgical tool of claim 1, wherein a longitudinal position of the suturing needle is locked relative to the end effector when the first and second grips are received in the grooves.

3. The surgical tool of claim 1, wherein a lateral position of the suturing needle is locked relative to the end effector when the first and second grips are received in the grooves.

4. The surgical tool of claim 1, wherein a radial position of the suturing needle is locked relative to the end effector when the first and second grips are received in the grooves.

5. The surgical tool of claim 1, wherein the grooves extend longitudinally and radially along a length of the suturing needle.

6. The surgical tool of claim 1, wherein the first and second grips comprise pyramidal grips.

7. A surgical tool, comprising:
an end effector providing opposing first and second jaws;
grips provided on the first jaw; and
a suturing needle defining grooves sized to receive the grips of the first jaw.

8. The surgical tool of claim 7, wherein a locking interface is defined between the first jaw and the suturing needle when the grips are received in the grooves.

9. The surgical tool of claim 7, wherein the grips are first grips and the surgical tool further comprises second grips provided on the second jaw.

10. The surgical tool of claim 9, wherein:
a first locking interface is defined between the first jaw and the suturing needle when the first grips are received in a first portion of the grooves; and
a second locking interface is defined between the second jaw and the suturing needle when the second grips are received in a second portion of the grooves.

11. The surgical tool of claim 10, wherein the first and second locking interfaces co-operatively lock a longitudinal position of the suturing needle relative to the end effector.

12. The surgical tool of claim 10, wherein the first and second locking interfaces co-operatively lock a lateral position of the suturing needle relative to the end effector.

13. The surgical tool of claim 10, wherein the first and second locking interfaces co-operatively lock a rotational position of the suturing needle relative to the end effector.

14. The surgical tool of claim 7, wherein the grooves extend radially and longitudinally along a length of the suturing needle.

15. A surgical tool, comprising:
an end effector providing opposing first and second jaws; and
a suturing needle graspable by the end effector, wherein:
a first locking interface is defined when the end effector grasps the suturing needle; and
a second locking interface is defined when the end effector grasps the suturing needle,
wherein the first and second locking interfaces co-operatively oppose multiple degrees of motion of the suturing needle relative to the end effector.

16. The surgical tool of claim 15, wherein the first and second locking interfaces co-operatively lock a longitudinal position of the suturing needle relative to the end effector.

17. The surgical tool of claim 15, wherein the first and second locking interfaces co-operatively lock a lateral position of the suturing needle relative to the end effector.

18. The surgical tool of claim 15, wherein the first and second locking interfaces co-operatively lock a rotational position of the suturing needle relative to the end effector.

19. The surgical tool of claim 15, wherein:
the first jaw provides first grips and the suturing needle provides first grooves to receive the first grips to define the first locking interface; and
the second jaw provides second grips and the suturing needle provides second grooves to receive the second grips to define the second locking interface.

20. The surgical tool of claim 19, wherein the first and second grooves extend radially and longitudinally along a length of the suturing needle.
